(19) United States
(12) Patent Application Publication
(10) Pub. No.: US 2010/0081681 A1
(43) Pub. Date: Apr. 1, 2010

(54) METHODS AND COMPOSITIONS FOR PREVENTING OR TREATING AGE-RELATED DISEASES

(76) Inventor: Mikhail V. Blagosklonny, Albany, NY (US)

Correspondence Address: MCDONNELL BOEHNEN HULBERT & BERGHOFF LLP, 300 S. WACKER DRIVE, 32ND FLOOR, CHICAGO, IL, 60606, US

(21) Appl. No.: 12/310,222

(22) PCT Filed: Aug. 16, 2007
(86) PCT No.: PCT/US07/76094

§ 371 Date: Nov. 18, 2009

Related U.S. Application Data
(60) Provisional application No. 60/837,859, filed on Aug. 16, 2006.
(60) Provisional application No. 60/878,638, filed on Jan. 5, 2007.

(51) Int. Cl. A61K 31/436; C12N 5/071; A61P 39/00

(52) U.S. Cl. 514/291; 435/375

(57) ABSTRACT

The invention provides methods for treating or preventing an age-related disease, condition, or disorder comprising administering a therapeutically effective amount of an inhibitor of TOR to a patient in need thereof. The invention also provides pharmaceutical compositions and topical formulations for treating or preventing an age-related disease, condition, or disorder comprising an inhibitor of TOR and a pharmaceutically acceptable carrier. In particular, the invention provides methods, pharmaceutical compositions, and topical formulations comprising rapamycin or an analog of rapamycin.

This application claims priority to U.S. Provisional Application Nos. 60/837,859, filed Aug. 16, 2006 and 60/878,638, filed Jan. 5, 2007, the disclosures of which are incorporated by reference herein.

### BACKGROUND OF THE INVENTION

1. Field of the Invention

The invention relates to methods for treating or preventing an age-related disease, condition, or disorder comprising administering a therapeutically effective amount of an inhibitor of TOR to a patient in need thereof. The invention also relates to pharmaceutical compositions and topical formulations for treating or preventing an age-related disease, condition, or disorder comprising an inhibitor of TOR and a pharmaceutically acceptable carrier. In particular, the invention relates to methods, pharmaceutical compositions, and topical formulations comprising rapamycin or an analog of rapamycin.

2. Background of the Invention

Cancer, prostate enlargement, cardiovascular diseases, stroke, atherosclerosis, hypertension, osteoporosis, insulin-resistance and type II diabetes, Alzheimer's disease, Parkinson's disease, and age-related macular degeneration are age-related diseases. Currently, these age-related diseases are treated separately. In other words, no single protocol has been identified that can be applied to the treatment of all age-related diseases. Moreover, because age-related diseases are investigated individually, even if a cure can be identified for one type of age-related disease (e.g., cancer), other age-related diseases (e.g., atherosclerosis) would continue to limit the maximal human life span, which has remained unchanged for years (Hayflick, 2000; Olshansky et al., 2005). It may be impossible to eliminate age-related diseases without eliminating the aging process per se. Thus, to prevent age-related diseases and also extend the maximal life span, it may be necessary to slow down the aging process. Unfortunately, as summarized recently, there is no known intervention that can slow the human aging process (Hayflick, 2000).

Rapamycin is a macrocyclic triene antibiotic produced by Streptomyces hygroscopicus. Rapamycin was found to have antifungal activity, particularly against Candida albicans, both in vitro and in vivo (Vezina et al., 1975; Baker et al., 1978; U.S. Pat. Nos. 3,929,992 and 3,993,749). Rapamycin has antitumor activity when administered either alone (U.S. Pat. No. 4,885,171) or in combination with picibanil (U.S. Pat. No. 4,401,653).

In addition, rapamycin has immunosuppressive effects (Thomson et al., 1989). Other macrocyclic molecules, such as Cyclosporin A and FK-506, are also known to be effective as immunosuppressive agents, and therefore, are useful in preventing transplant rejection (Thomson et al., 1989; Calne et al., 1978; and U.S. Pat. No. 5,100,899). Mattel et al. disclosed that rapamycin is effective in the experimental allergic encephalomyelitis model (a model for multiple sclerosis) and the adjuvant arthritis model (a model for rheumatoid arthritis), and effectively inhibited the formation of IgE-like antibodies.

Rapamycin can also be used to prevent or treat systemic lupus erythematosus (U.S. Pat. No. 5,078,999), pulmonary inflammation (U.S. Pat. No. 5,080,899), insulin dependent diabetes mellitus (U.S. Pat. No. 5,321,009), skin disorders such as psoriasis (U.S. Pat. No. 5,286,730), bowel disorders (U.S. Pat. No. 5,286,731), smooth muscle cell proliferation and intimal thickening following vascular injury (U.S. Pat. Nos. 5,288,711 and 5,516,781), adult T-cell leukemia/lymphoma (European Patent Application No. EP 0 525 960 A1), ocular inflammation (U.S. Pat. No. 5,387,589), malignant carcinomas (U.S. Pat. No. 5,206,018), cardiac inflammatory disease (U.S. Pat. No. 5,496,832), and anemia (U.S. Pat. No. 5,561,138).

The use of rapamycin has also been suggested for treating psoriasis (Marsland et al., 2002), skin keloids and scars (Ong et al., 2007), multiple sclerosis (Farrell et al., 2005), and arthritis (Carlson et al., 1993; Foroncewicz et al., 2005).

Rapamycin 42-ester with 3-hydroxy-2-(hydroxymethyl)-2-methylpropionic acid (CCI-779) is an ester of rapamycin which has demonstrated significant inhibitory effects on tumor growth in both in vitro and in vivo models. The preparation and use of hydroxyesters of rapamycin, including CCI-779, are disclosed in U.S. Pat. Nos. 5,362,718 and 6,277,983.

Metformin is an anti-diabetic agent that increases insulin sensitivity. Recently, metformin has been shown to activate the LKB1/AMPK pathway, thus inhibiting the “target of rapamycin” (TOR) (Shaw et al., 2005). Inhibition of TOR restores insulin sensitivity, thus explaining the anti-diabetic effects of metformine. Notably, metformin and its analog phenformin extend life span in rodents (Anisimov et al., 2005a; Anisimov et al., 2005b).

Other references disclosing the use of rapamycin, rapamycin analogs, or mTOR inhibitors for treating or preventing various diseases, disorders, or conditions include:

U.S. Pat. No. 6,187,756, which discloses compositions and methods for treating neurological disorders and neurodegenerative diseases.

U.S. Pat. No. 7,026,330, which discloses methods for treating patients having an early B cell derived acute lymphoblastic leukemia with rapamycin or a rapamycin derivative, either alone or in combination with an IL-7 inhibitor.

U.S. Pat. No. 7,083,802, which discloses formulations for treating ocular conditions such as dry eye disease by administering rapamycin and/or ascomycin intraocularly.

U.S. Patent Application Publication No. 2002/0183239, published Dec. 5, 2002, which discloses the use of a combination of an mTOR inhibitor and an antimetabolite antineoplastic agent in the treatment of neoplasms. U.S. Patent Application Publication No. 2004/0176339, published Sep. 9, 2004, which discloses the use of a combination of CCI-779 and an aromatase inhibitor in the treatment of neoplasms.

U.S. Patent Application Publication No. 2004/0258662, published Dec. 23, 2004, which discloses the use of a combination of CCI-779 and interferon alpha in the treatment of neoplasms.

U.S. Patent Application Publication No. 2005/0026893, published Feb. 3, 2005, which discloses methods of treating diseases using HSP90-inhibiting agents in combination with immunosuppressants.

U.S. Patent Application Publication No. 2005/0070567, published Mar. 31, 2005, which discloses methods of diagnosing and treating disorders such as tuberous sclerosis, which are caused by mutations in the TSC genes, and methods and compositions for treating cancers mediated by TSC signaling disorders.

U.S. Patent Application Publication No. 2005/0187241, published Aug. 25, 2005, which discloses the use of rapamycin to inhibit CNV and wet AMD.

U.S. Patent Application Publication No. 2006/0035904, published Feb. 16, 2006, which discloses the use of a combination of an mTOR inhibitor and an antimetabolite antineoplastic agent in the treatment of neoplasms.

U.S. Patent Application Publication No. 2006/0035907, published Feb. 16, 2006, which discloses methods of treating abnormal cell growth in a mammal, such as a human, by administering to the mammal a therapeutically effective amount of a c-MET inhibitor and a mammalian target of rapamycin (mTOR) inhibitor.

U.S. Patent Application Publication No. 2006/0094674, published May 4, 2006, which discloses methods and compositions including an mTOR inhibitor, a tyrosine kinase inhibitor, and optionally an MEK inhibitor for reducing the proliferation of and enhancing the apoptosis of neoplastic cells.

U.S. Patent Application Publication No. 2006/0135549, published Jun. 22, 2006, which discloses the use of rapamycin analogues in the treatment of neurological, proliferative, and inflammatory disorders.

U.S. Patent Application Publication No. 2006/0173033, published Aug. 3, 2006, which discloses the use of rapamycin and rapamycin derivatives for the treatment of bone loss.

U.S. Patent Application Publication No. 2006/0182771, published Aug. 17, 2006, which discloses methods for treating AMD using self-emulsifying formulations of rapamycin.

U.S. Patent Application Publication No. 2006/0247265, published Nov. 2, 2006, which discloses the methods for treating disorders of the eye, including AMD, using inhibitors of mTOR, including rapamycin and rapamycin analogs.

U.S. Patent Application Publication No. 2006/0263409, published Nov. 23, 2006, which discloses methods for treating disorders of the eye, including AMD, using topical and intraocular applications of rapamycin.

U.S. Patent Application Publication No. 2006/0264453, published Nov. 23, 2006, which discloses methods of treating or preventing diseases or conditions, such as choroidal neovascularization, wet AMD, and dry AMD, and preventing transition of dry AMD to wet AMD, using the liquid rapamycin formulations.

U.S. Patent Application Publication No. 2007/0059336, published Mar. 15, 2007, which discloses anti-angiogenic sustained release intraocular implants and methods for using such implants.

U.S. Patent Application Publication No. 2007/0099844, published May 3, 2007, which discloses compositions and methods for the treatment of malignancy and chronic viral infection.

U.S. Patent Application Publication No. 2007/0104721, published May 10, 2007, which discloses methods and kits for treatment of metastatic breast cancer, using herceptin, temsirolimus, and/or HKI-272, optionally in combination with other anti-neoplastic agents, or immune modulators.

U.S. Patent Application Publication No. 2007/0105761, published May 10, 2007, which discloses methods, compositions, and kits for the treatment of ophthalmic disorders, including AMD, wherein the compositions comprise a corticosteroid in combination with a non-steroidal immunosuppressant, including rapamycin.

U.S. Patent Application Publication No. 2007/0116774, published May 24, 2007, which discloses methods and compositions for treating proliferative diseases using nanoparticles.

U.S. Patent Application Publication No. 2007/0155771, published Jul. 5, 2007, which discloses methods and uses of autophagy inducing agents, such as rapamycin macrolides, in the treatment of Protein Conformational Disorders.

### SUMMARY OF THE INVENTION

The present invention provides methods for treating or preventing an age-related disease, condition, or disorder comprising administering a therapeutically effective amount of an inhibitor of TOR to a patient in need thereof.

The present invention also provides pharmaceutical compositions for treating or preventing an age-related disease, condition, or disorder comprising an inhibitor of TOR and a pharmaceutically acceptable carrier.

The present invention also provides topical formulations for treating or preventing an age-related disease, condition, or disorder comprising an inhibitor of TOR and a pharmaceutically acceptable carrier.

Specific preferred embodiments of the present invention will become evident from the following more detailed description of certain preferred embodiments and the claims.

### BRIEF DESCRIPTION OF THE DRAWINGS

FIG. 1 shows the effect of growth factors (GF) on the Raf-1/MEK/ERK and PI-3K/Akt signal transduction pathways.

FIG. 2 shows the results of doxorubicin (DOX) exposure in WI-38 fibroblasts.

FIG. 3 shows that rapamycin prevents senescence-associated beta-galactosidase (beta-Gal) activity; WI-38 normal human fibroblasts were untreated (control) or treated with 100 nM doxorubicin (dox) or 150 μM H₂O₂ in the presence or absence of 100 nM rapamycin (rapa), and the percentage of beta-gal-positive cells (beta-Gal %) was determined.

FIG. 4 shows that rapamycin decreases a senescence-associated increase in cellular protein; WI-38 normal human fibroblasts were untreated (control) or treated with 100 nM doxorubicin (dox) or 150 μM H₂O₂ in the presence or absence of 100 nM rapamycin (rapa), and the amount of protein per cell was determined.

FIG. 5 shows that rapamycin prevents irreversible cell arrest (i.e., cell senescence); WI-38 normal human fibroblasts were pre-treated with 150 μM H₂O₂ or 150 μM H₂O₂ plus rapamycin (rapa) in 18 parallel plates for three days, the plates were washed twice with fresh medium, and the cells on each plate were counted following the wash and at 1, 2, 3, 4, and 5 days after washing.

FIG. 6 shows that rapamycin prevents irreversible loss of clonogenity associated with senescence; WI-38 normal human fibroblasts were untreated (control) or pre-treated with 150 μM H₂O₂ or 150 μM H₂O₂ plus rapamycin (rapa) for three days, the cells were washed twice with fresh medium, trypsinized and counted, 100 cells were plated onto 100 mm plates, and the number of cell colonies per plate was counted after fourteen days.

FIG. 7 shows the relationship between TOR and a number of genes that modulate the TOR pathway.

FIG. 8 shows a schematic representation of the relationship between TOR modulation and human age-related diseases.

### DETAILED DESCRIPTION OF THE INVENTION

Age-related diseases are the main causes of death for people older than 45 years of age, and their incidence is dramatically increased with age. Age-related diseases include common cancer, prostate enlargement, cardiovascular diseases, stroke, atherosclerosis, hypertension, osteoporosis, type II diabetes, Alzheimer's disease, Parkinson's disease, and age-related macular degeneration. Currently, age-related diseases are treated separately. The invention provides a regimen that can be applied to the treatment of most, if not all, age-related diseases. In particular, the methods of the invention are directed to the inhibition or retardation of the aging process, which, in turn, results in the delay, prevention, or treatment of age-related diseases.

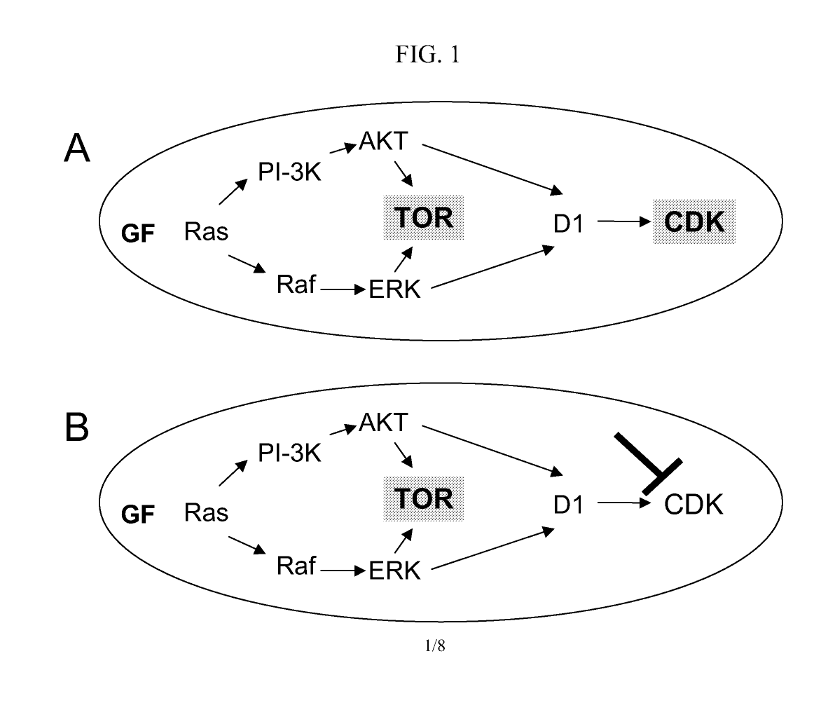

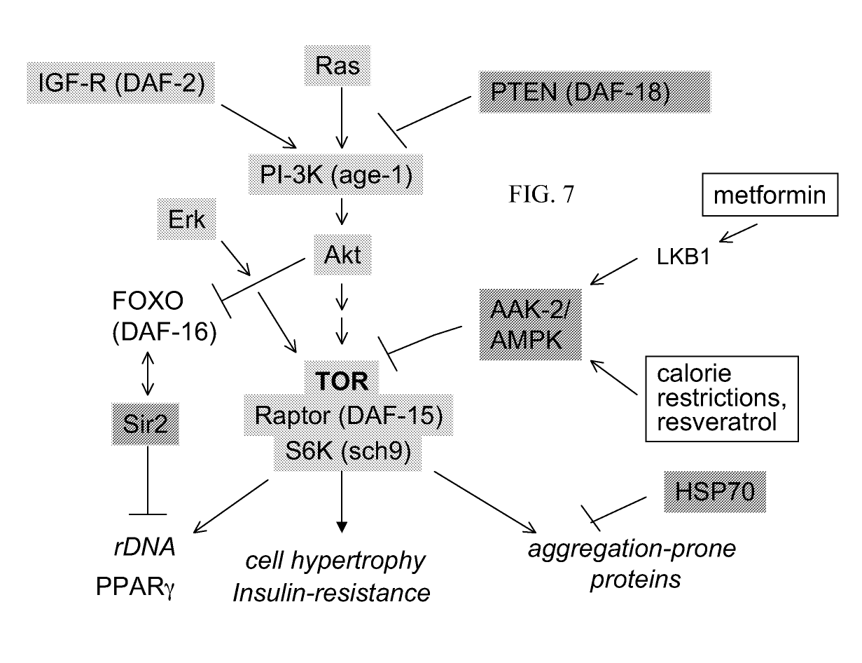

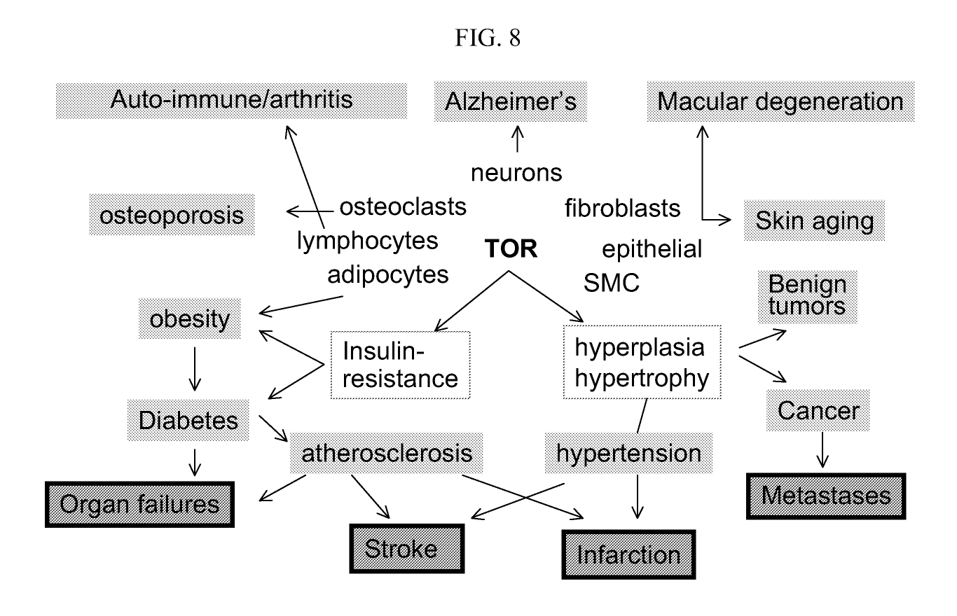

The invention is based on the merger of three independent fields of research: (1) cell senescence (see FIGS. 1-6), (2) genetic control of longevity (from yeast to mammals) (see FIG. 7), and (3) human age-related diseases (see FIG. 8; Blagosklonny, 2006a; Blagosklonny, 2006b; Blagosklonny, 2007). Prior to the invention, there was no known way to inhibit or retard the aging process. The invention takes advantage of the recognition that the “target of rapamycin” (TOR) pathway is involved in cell aging and senescence, organism aging and longevity, and age-related diseases. In particular, cell aging and senescence, organism aging and longevity, and age-related diseases are caused by or associated with activation of the TOR pathway.

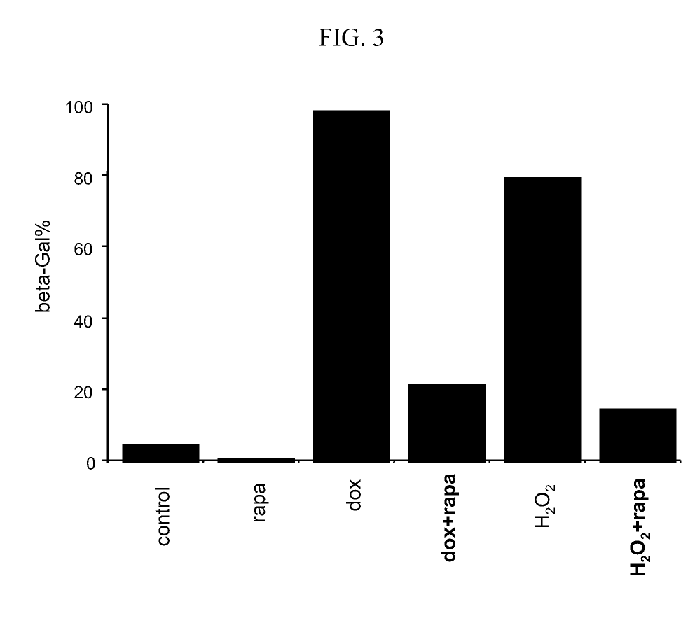

For example, FIGS. 1-6 show that the TOR pathway is involved in human cell aging. The TOR pathway has also been shown to be the main accelerator of animal aging and human diseases (Blagosklonny, 2007). FIGS. 3-6 show that rapamycin—a non-toxic inhibitor of TOR—blocks accelerated aging of human cells. In addition, a retrospective analysis of clinical data indicates that rapamycin can be used to prevent cancer and osteoporosis (i.e., age-related diseases) in humans (Blagosklonny, 2007). Thus, rapamycin can be used in the methods of the invention to delay, prevent, or treat age-related diseases and prolong human life span by inhibit aging.

The claimed invention takes advantage of the recognition that TOR is involved in cell aging, organism longevity, and age-related diseases of aging. In addition, the “side effects” observed following the administration of rapamycin are consistent with its anti-aging effects. For example, historically, renal transplant patients often developed tumors—particularly Kaposi's sarcoma—following the administration of immunosuppressants. When rapamycin (sirolimus) was added to immunosuppressive regimens in 1997, it was unexpectedly found to prevent tumors in renal transplant patients (Yakupoglu et al., 2006; Nungaray et al., 2005; Kauffman et al., 2005; Campistol et al., 2006; Mathew et al., 2004). In fact, rapamycin not only prevented new tumors from developing, it also resulted in the regression of pre-existing tumors (Zmonarski et al., 2005; Mohsin et al., 2005; Cullis et al., 2006; Rizell et al., 2005; Stallone et al., 2005).

In addition to rapamycin's anti-tumorigenic capability, the most common side effect of rapamycin administration is the increase of blood lipids (hyperlipidemia or hypertriglyceridemia) such as triglycerides (Kahan, 2004; Rodriguez et al., 2006). Thus, rapamycin mobilizes lipids from the fat tissue into the blood and prevents the accumulation of lipids in tissues including the vascular wall. The mobilization of fat from fat tissue results in hypertriglyceridemia (Morrisett et al., 2002), which is the cause of rapamycin's calorie-restriction-mimetic effect (Blagosklonny, 2006a). Calorie restriction has been shown to extend life span in a variety of species ranging from yeast to mice.

### A. Relationship Between the TOR Pathway and Cell Senescence

Growth factors (GF) activate the Raf-1/MEK/ERK and PI-3K/Akt pathways (see FIG. 1). These pathways, in turn, activate TOR, which stimulates protein synthesis and cell growth, i.e., cell mass and cell size (Blagosklonny, 2006b; Blagosklonny, 2007). During cell senescence, however, while the cell cycle is blocked, the growth-factor promoting pathways are not (see FIG. 1B). Moreover, eIF-4E, a downstream effector of TOR is known to induce cell senescence (Ruggero et al., 2004). Thus, while oncogenic Ras, Raf-1, B-Raf, MEK, Akt, and PI-3K all activate TOR, each also may cause cycle arrest, leading to cell senescence. Since it is TOR that stimulates cell growth, it is possible that TOR activity is necessary for acquiring the senescent phenotype (Blagosklonny, 2006b).

Activation of the TOR pathway also results in the activation of ribosomal DNA transcription, ribosome biogenesis, mitogen and VEGF secretion, a large cell morphology, protein synthesis and cell growth, HIF-1 induction and secondary insulin resistance, and growth factor resistance (see Blagosklonny, 2006a; Blagosklonny, 2006b; Blagosklonny, 2007). Senescent cells are distinguished by their large cell morphology, large nucleus, flat appearance, and secretion of proteases, matrix, mitogens, VEGF, and other biologically active molecules (Krtolica et al., 2001; Campisi, 2005). Cellular hypertrophy can in turn be connected to hallmarks of aging (such as skin wrinkles, prostate enlargement in men, and atherosclerotic plaques) (see Blagosklonny, 2006a).

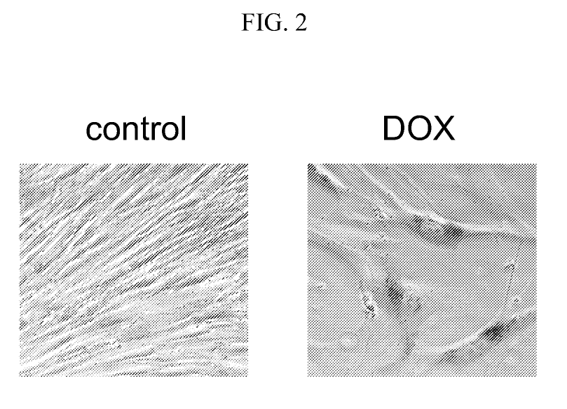

To illustrate the relationship between the TOR pathway and cell senescence, experiments were performed using WI-38 fibroblasts, a cell line that is commonly used to investigate cell senescence (Sarraj et al., 2001). In WI-38 fibroblasts, exposure to doxorubicin (DOX) induces accelerated cell senescence (see FIG. 2). Cell senescence is characterized by a large and flat cell morphology, beta-galactosidase expression in the cytoplasm as a terminal, non-replicative condition. FIGS. 3-6 confirm that rapamycin, which inhibits TOR, also inhibits cell senescence.

FIG. 3 shows that rapamycin prevents senescence-associated beta-galactosidase (beta-Gal) activity. In particular, WI-38 normal human fibroblasts were treated with 100 nM doxorubicin (dox) or 150 μM H₂O₂ or left untreated (control) in the presence or absence of 100 nM rapamycin (rapa). After three days, cells were stained for beta-Gal, and the percentage of beta-gal-positive cells (i.e., senescent cells) was determined. Exposure to rapamycin decreased a number of senescent cells.

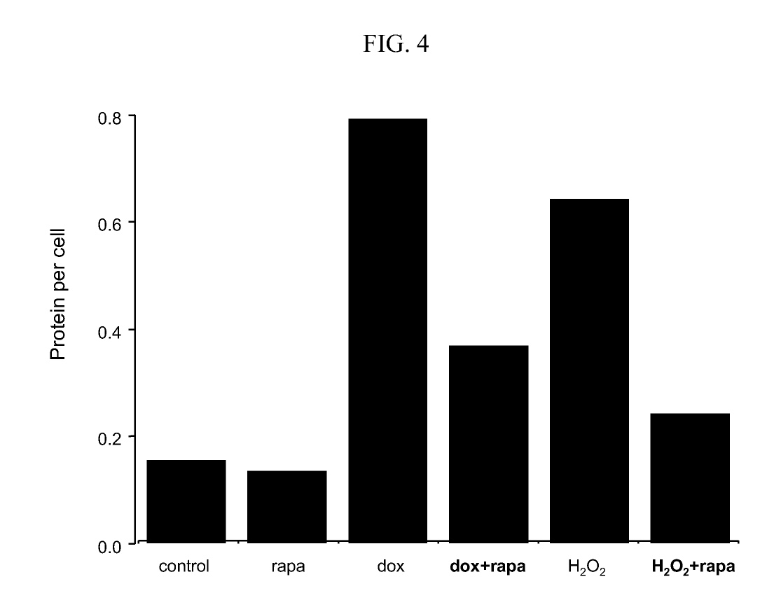

FIG. 4 shows that rapamycin decreases a senescence-associated increase in cellular protein. In particular, WI-38 normal human fibroblasts were treated with 100 nM doxorubicin (dox) or 150 μM H₂O₂ or left untreated (control) in the presence or absence of 100 nM rapamycin (rapa). After three days, the number of cells and protein content were measured, and the amount of protein per cell was calculated. Exposure to rapamycin decreased the senescence-associated increase of cellular protein.

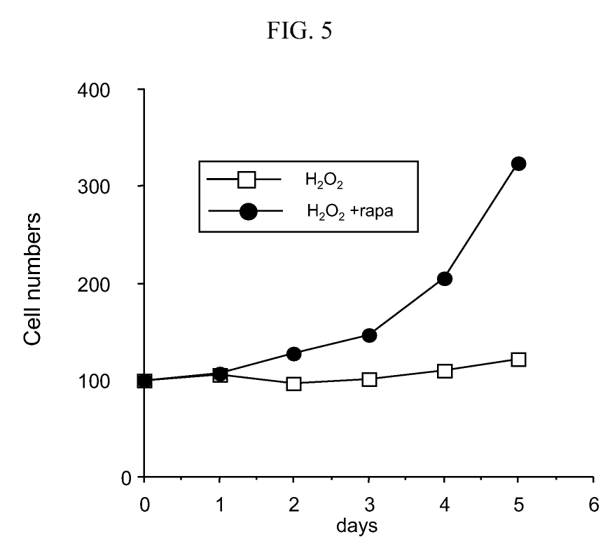

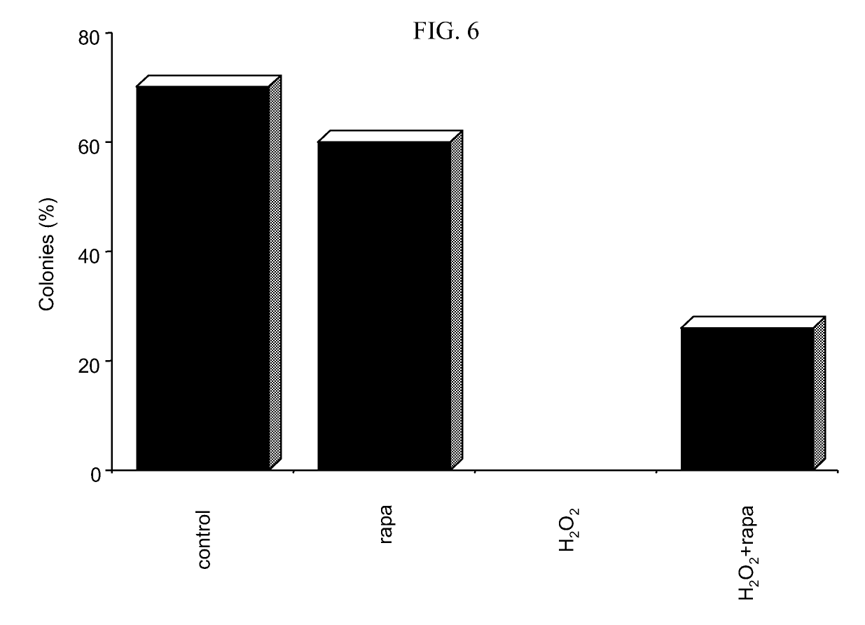

FIG. 5 shows that rapamycin prevents irreversible cell arrest (i.e., cell senescence). In particular, WI-38 normal human fibroblasts were pre-treated with 150 μM H₂O₂ or 150 μM H₂O₂ plus rapamycin (rapa) in 18 parallel plates. After three days, the plates were washed twice with fresh medium to remove the drugs, and the cells on each plate were counted (day 0). The cells on each plate were also counted at 1, 2, 3, 4, and 5 days after washing. As shown in FIG. 6, when cells were pre-treated with H₂O₂ alone, they did not regain proliferative capacity. However, when cells were pre-treated with H₂O₂ in the presence of rapamycin, the cells were able to recover and to start proliferation upon removal of the H₂O₂ and rapamycin.

FIG. 6 shows that rapamycin prevents irreversible loss of clonogenity associated with senescence. In particular, WI-38 normal human fibroblasts were pre-treated with 150 μM H₂O₂ or 150 μM H₂O₂ plus rapamycin (rapa) or left untreated (control). After three days, the plates were washed twice with fresh medium to remove the drugs, the cells were trypsinized and counted, and 100 cells were plated onto 100 mm plates. After fourteen days, the number of cell colonies per plate was counted. As shown in FIG. 7, when the cells were treated with H₂O₂ alone, they lost clonogenic activity. However, when cells were treated with H₂O₂ in the presence of rapamycin, they were able to recover and to form colonies upon removal of the H₂O₂ and rapamycin.

Thus, as shown in FIGS. 3-6, rapamycin blocks all three hallmarks of cell senescence: beta-galactosidase activity, cell hypertrophy (amount of protein per cell), and irreversible cell cycle arrest.

### B. Relationship Between the TOR Pathway and Prolonged Life Span

There are numerous genes whose inactivation results in prolonged life span. For example, the deletion of TOR1 extends lifespan in yeast (Kaeberlein et al., 2005). Also, in yeast, worms, flies, and mice, mutations in genes that encode proteins which inhibit TOR prolong lifespan, while mutations in genes that encode proteins which activate TOR shorten lifespan (Kaeberlein et al., 2005; Kapahi et al., 2004; Powers et al., 2006; Jia et al., 2004; Apfeld et al., 2004; Kimura et al., 1997; Vellai et al., 2003; Berdichevsky et al., 2006; Luong et al., 2006; Bartke, 2006; Sharp et al., 2005; Um et al., 2004). In addition, in humans, reduced insulin/IGF-1 signaling and increased sensitivity to insulin have been shown to be associated with extreme longevity (van Heemst et al., 2005; Paolisso et al., 2001). Thus, a number of genes in a variety of species are known to modulate the TOR pathway either upstream or downstream of TOR (see FIG. 7). The methods and compositions of the invention take advantage of known associations between specific genes and the TOR pathway (see FIG. 7).

### C. Relationship Between the TOR Pathway and Age-Related Diseases

An association between TOR modulation and human age-related disease has been established for a number of age-related diseases (see FIG. 8). Among diseases for which TOR involvement has been demonstrated are the following:

1. Benign Prostatic Hyperplasia

Benign prostatic hyperplasia (BPH), which is also known as nodular hyperplasia, benign prostatic hypertrophy, or benign enlargement of the prostrate (BEP), refers to the increase in size of the prostrate in middle-aged and elderly men. This disorder is characterized by an accumulation of senescent (enlarged) cells (Choi et al., 2000; Bavik et al., 2006). The methods of the invention, therefore, could be used to normalize cell size, and thereby reduce prostate enlargement.

2. Metabolic Syndrome: Insulin Resistance and its Complications

Metabolic syndrome is characterized by obesity (especially abdominal obesity) and insulin resistance with elevated plasma insulin and glucose, elevated blood pressure, increased propensity to thrombosis, and a pro-inflammatory state. Metabolic syndrome is associated with aging and TOR signaling (Le Bacquer et al., 2007).

In addition, TOR inactivates insulin signaling via S6K, causing insulin-resistance (Tzatsos et al., 2006; Um et al., 2004; Khamzina et al., 2005; Zhang et al., 2007; Zhang et al., 2003; Manning, 2004; Shah et al., 2004; Harrington et al., 2005). TOR is also known to be associated with complications of diabetes. For example, the activation of TOR causes renal hypertrophy during the early stages of diabetes (Sakaguchi et al., 2006).

3. Hypertension and Atherosclerosis

In hypertension, a thickening of the arteries occurs through smooth muscle cell (SMC) hypertrophy, which depends on the Akt/TOR/S6K pathway (Haider et al., 2002). The methods of the invention, therefore, could be used to slow the progression of hypertension. For example, in animal models, the systemic administration of rapamycin reduces neointimal thickening and slows the progression of atherosclerosis in apoE-deficient mice having elevated levels of cholesterol (Elloso et al., 2003).

4. Cardiac Hypertrophy

Over-activation of PI-3K/TOR has been shown to cause cardiac hypertrophy (Proud, 2004; Oudit et al., 2004; Ha et al., 2005).

5. Osteoporosis

Osteoporosis is caused by bone-resorbing osteoclasts. TOR expression increases osteoclast activity, thus causing osteoporosis (Kneissel et al., 2004).

6. Neurodegenerative Diseases

TOR stimulates the synthesis of aggregate-prone proteins. Intracellular aggregate-prone proteins, in turn, contribute to neurodegenerative diseases such as Alzheimer's disease, Parkinson's disease, and Huntington's disease (Rubinsztein, 2006). Moreover, TOR causes neurodegeneration in a Drosophila tauopathy model (Khurana et al., 2006), and rapamycin has been shown to enhance the autophagic clearance of pathologic proteins, thereby reducing their toxicity (Berger et al., 2006).

With respect to specific neurodegenerative diseases, the TOR pathway has been shown to be involved with Alzheimer's disease by increasing Tau protein synthesis (Li et al., 2005; An et al., 2003). In addition, a correlation between activated TOR in blood lymphocytes and memory and cognitive decline has been established in individuals suffering from Alzheimer's disease (Paccalin et al., 2006). Furthermore, rapamycin has been suggested for the treatment of Parkinson's disease (Rubinsztein, 2006).

In one embodiment of the invention, the aging process is inhibited or retarded by administering a therapeutically effective amount of an inhibitor of the TOR pathway (a TOR inhibitor) to a patient. In this manner, cell aging, organism aging, or age-related diseases are treated or prevented. Thus, the present invention provides methods for treating or preventing an age-related disease, condition, or disorder comprising administering a therapeutically effective amount of an inhibitor of TOR to a patient in need thereof. Inhibitors of TOR are molecular agents that directly or indirectly decrease the activity of TOR. Direct inhibitors of TOR include rapamycin and its analogs. Indirect inhibitors include metformin and resveratrol (see Blagosklonny, 2007). Suitable inhibitors of TOR include, but are not limited to metformin, rapamycin, everolimus, tacrolimus, CCI-779, ABT-578, AP-23675, AP-23573, AP-23841, 7-epi-rapamycin, 7-thiomethyl-rapamycin, 7-epi-trimethoxyphenyl-rapamycin, 7-epi-thiomethyl-rapamycin, 7-demethoxy-rapamycin, 32-demethoxy-rapamycin, 2-desmethyl-rapamycin, or 42-O-(2-hydroxy)ethyl rapamycin.

In one embodiment of the methods of the invention, the inhibitor of TOR is rapamycin or an analog of rapamycin. Suitable analogs of rapamycin include, but are not limited to, everolimus, tacrolimus, CCI-779, ABT-578, AP-23675, AP-23573, AP-23841, 7-epi-rapamycin, 7-thiomethyl-rapamycin, 7-epi-trimethoxyphenyl-rapamycin, 7-epi-thiomethyl-rapamycin, 7-demethoxy-rapamycin, 32-demethoxy-rapamycin, 2-desmethyl-rapamycin, or 42-O-(2-hydroxy)ethyl rapamycin.

The methods of the invention may be used to treat or prevent age-related diseases, conditions, or disorders such as insulin resistance (i.e., impaired glucose tolerance), benign prostatic hyperplasia, hearing loss, osteoporosis, age-related macular degeneration, neurodegenerative diseases such as Alzheimer's disease or Parkinson's disease, a skin disease, or aging skin. In one embodiment of the methods of the invention, the age-related disease, condition, or disorder is a skin disease. Examples of skin diseases for which the methods of the invention may be used include seborreic keratosis, actinic keratosis, keloid, psoriasis, and Kaposi's sarcoma. In another embodiment of the methods of the invention, the age-related disease, condition, or disorder is an aging skin condition. Examples of aging skin conditions for which the methods of the invention may be used include age-related spots, pigment spots, wrinkles, photo-aged skin, or angiogenic spots. In still another embodiment of the methods of the invention, the inhibitor of TOR is administered to extend an individual's life span.

The methods of the invention may be used to inhibit cellular or organismal events. In one embodiment of the invention, the cellular event being inhibited is cell aging. In another embodiment of the invention the cellular event being inhibited is cell hypertrophy. In still another embodiment of the invention, the cellular event being inhibited is organism aging.

In order to maximize the effects of the inhibitor of TOR and decrease its side effects, in one embodiment of methods of the invention, the inhibitor of TOR is administered with a second compound. Suitable compounds that may be administered with the inhibitor of TOR include, but are not limited to, a vitamin such as vitamin E or vitamin A; an antibacterial antibiotic; an antioxidant (i.e., an inhibitor of free radicals); L-carnitine; lipoic acid; metformine; resveratrol; leptine; a non-steroid anti-inflammatory drug, such as aspirin or acetaminophen; a bone resorption inhibitor; and a COX inhibitor.

In the methods of the invention, the inhibitor of TOR may be administered in the form of a pill, tablet, solution, cream, liniment, eye drop, ear drop, or ear cream. Other forms of administration are also encompassed by the methods of the invention. Those skilled in the art would understand how best to deliver the inhibitor of TOR depending on the age-related disease, condition, or disorder to be treated or prevented.

In the methods of the invention the inhibitor of TOR may be administered orally, topically, or by injection. Other means of administration are also encompassed by the methods of the invention. Those skilled in the art would understand the best means for delivering the inhibitor of TOR depending on the age-related disease, condition, or disorder to be treated or prevented.

The present invention provides topical formulations for treating or preventing an age-related disease, condition, or disorder comprising an inhibitor of TOR and a pharmaceutically acceptable carrier. Suitable inhibitors of TOR include, but are not limited to metformin, rapamycin, everolimus, tacrolimus, CCI-779, ABT-578, AP-23675, AP-23573, AP-23841, 7-epi-rapamycin, 7-thiomethyl-rapamycin, 7-epi-trimethoxyphenyl-rapamycin, 7-epi-thiomethyl-rapamycin, 7-demethoxy-rapamycin, 32-demethoxy-rapamycin, 2-desmethyl-rapamycin, or 42-O-(2-hydroxy)ethyl rapamycin.

In one embodiment of the topical formulations of the invention, the inhibitor of TOR is rapamycin or an analog of rapamycin. Suitable analogs of rapamycin include, but are not limited to, everolimus, tacrolimus, CCI-779, ABT-578, AP-23675, AP-23573, AP-23841, 7-epi-rapamycin, 7-thiomethyl-rapamycin, 7-epi-trimethoxyphenyl-rapamycin, 7-epi-thiomethyl-rapamycin, 7-demethoxy-rapamycin, 32-demethoxy-rapamycin, 2-desmethyl-rapamycin, or 42-O-(2-hydroxy)ethyl rapamycin. In preparing the topical formulations of the invention, those skilled in the art would be able to determine a suitable concentration for the inhibitor of TOR.

The topical formulations of the invention include, but are not limited to, creams, ointments, emulsions, gels, and lotions. In one embodiment of the topical formulations of the invention, the topical formulation comprises at least one inert material (such as an oil) in addition to the inhibitor of TOR. In one embodiment of the topical formulations of the invention, the ingredients of the topical formulation are provided in a moisturizing cream base. Preservatives may also be provided in the topical formulations of the invention to increase the formulation's shelf life. Those skilled in the art would understand how to modify the topical formulations of the invention by adding additional active ingredients or inert materials.

The topical formulations of the invention may be used to treat or prevent age-related diseases, conditions, or disorders such as a skin disease or aging skin. Examples of skin diseases for which the topical formulations of the invention may be used include seborreic keratosis, actinic keratosis, keloid, psoriasis, and Kaposi's sarcoma. Because seborrhoeic keratosis is caused by an accumulation of senescent epidermal cells (Nakamura et al., 2003), a preferred skin disease for which the topical formulations of the invention may be used is seborrhoeic keratosis. Examples of aging skin conditions for which the topical formulations of the invention may be used include age-related spots, pigment spots, wrinkles, photo-aged skin, or angiogenic spots. The topical formulations of the invention may also be used for skin rejuvenation.

In order to maximize the effects of the inhibitor of TOR and decrease its side effects, in one embodiment of topical formulations of the invention, the topical formulation may comprise a second compound. Suitable compounds that may be used with the inhibitor of TOR include, but are not limited to, a vitamin such as vitamin E or vitamin A; an antibacterial antibiotic; an antioxidant (i.e., an inhibitor of free radicals; L-carnitine; lipoic acid; metformine; resveratrol; leptine; a non-steroid anti-inflammatory drug, such as aspirin or acetaminophen; and a COX inhibitor.

The present invention provides pharmaceutical compositions for treating or preventing an age-related disease, condition, or disorder comprising an inhibitor of TOR and a pharmaceutically acceptable carrier. Suitable inhibitors of TOR include, but are not limited to rapamycin, everolimus, tacrolimus, CCI-779, ABT-578, AP-23675, AP-23573, AP-23841, 7-epi-rapamycin, 7-thiomethyl-rapamycin, 7-epi-trimethoxyphenyl-rapamycin, 7-epi-thiomethyl-rapamycin, 7-demethoxy-rapamycin, 32-demethoxy-rapamycin, 2-desmethyl-rapamycin, or 42-O-(2-hydroxy)ethyl rapamycin.

In one embodiment of the pharmaceutical compositions of the invention, the inhibitor of TOR is rapamycin or an analog of rapamycin. Suitable analogs of rapamycin include, but are not limited to, everolimus, tacrolimus, CCI-779, ABT-578, AP-23675, AP-23573, AP-23841, 7-epi-rapamycin, 7-thiomethyl-rapamycin, 7-epi-trimethoxyphenyl-rapamycin, 7-epi-thiomethyl-rapamycin, 7-demethoxy-rapamycin, 32-demethoxy-rapamycin, 2-desmethyl-rapamycin, or 42-O-(2-hydroxy)ethyl rapamycin. In preparing the pharmaceutical compositions of the invention, those skilled in the art would be able to determine a suitable concentration for the inhibitor of TOR.

The pharmaceutical compositions of the invention may be used to treat or prevent age-related diseases, conditions, or disorders such as insulin resistance (i.e., impaired glucose tolerance), benign prostatic hyperplasia, hearing loss, osteoporosis, age-related macular degeneration, neurodegenerative diseases such as Alzheimer's disease or Parkinson's disease, a skin disease, or aging skin. In one embodiment of the pharmaceutical compositions of the invention, the age-related disease, condition, or disorder is a skin disease. Examples of skin diseases for which the pharmaceutical compositions of the invention may be used include seborreic keratosis, actinic keratosis, keloid, psoriasis, and Kaposi's sarcoma. In another embodiment of the pharmaceutical compositions of the invention, the age-related disease, condition, or disorder is an aging skin condition. Examples of aging skin conditions for which the pharmaceutical compositions of the invention may be used include age-related spots, pigment spots, wrinkles, photo-aged skin, or angiogenic spots.

In order to maximize the effects of the inhibitor of TOR and decrease its side effects, in one embodiment of pharmaceutical compositions of the invention, the pharmaceutical composition may comprise a second compound. Suitable compounds that may be used with the inhibitor of TOR include, but are not limited to, a vitamin such as vitamin E or vitamin A; an antibacterial antibiotic; an antioxidant (i.e., an inhibitor of free radicals; L-carnitine; lipoic acid; metformine; resveratrol; leptine; a non-steroid anti-inflammatory drug, such as aspirin or acetaminophen; a bone resorption inhibitor; and a COX inhibitor. Those skilled in the art would understand how to modify the pharmaceutical compositions of the invention by adding additional active ingredients or inert materials.

It should be understood that the foregoing disclosure emphasizes certain specific embodiments of the invention and that all modifications or alternatives equivalent thereto are within the spirit and scope of the invention as set forth in the appended claims. All references cited herein are incorporated by reference in their entirety, for all purposes.

### BIBLIOGRAPHY

1. An et al., “Up-regulation of phosphorylated/activated p70 S6 kinase and its relationship to neurofibrillary pathology in Alzheimer's disease,” Am. J. Pathol. 163: 591-607 (2003).
2. Anisimov et al., “Effect of metformin on life span and on the development of spontaneous mammary tumors in HER-2/neu transgenic mice,” Exp. Gerontol. 40: 685-93 (2005a).
3. Anisimov et al., “Metformin decelerates aging and development of mammary tumors in HER-2/neu transgenic mice,” Bull. Exp. Biol. Med. 139: 721-23 (2005b).
4. Apfeld et al., “The AMP-activated protein kinase AAK-2 links energy levels and insulin-like signals to lifespan in C. elegans,” Genes Dev. 18: 3004-09 (2004).
5. Baker et al., “Rapamycin (AY-22,989), a new antifungal antibiotic. III. In vitro and in vivo evaluation,” J. Antibiot. 31: 539-45 (1978).
6. Bartke, “Long-lived Klotho mice: new insights into the roles of IGF-1 and insulin in aging,” Trends Endocrinol. Metab. 17: 33-35 (2006).
7. Bavik et al., “The gene expression program of prostate fibroblast senescence modulates neoplastic epithelial cell proliferation through paracrine mechanisms,” Cancer Res. 66: 794-802 (2006).
8. Berdichevsky et al., “A stress response pathway involving sirtuins, forkheads and 14-3-3 proteins,” Cell Cycle 5: 2588-91 (2006).
9. Berger et al., “Rapamycin alleviates toxicity of different aggregate-prone proteins,” Hum. Mol. Genet. 15: 433-42 (2006).
10. Blagosklonny, “Aging and immortality: quasi-programmed senescence and its pharmacologic inhibition,” Cell Cycle 5: 2087-102 (2006a).
11. Blagosklonny, “Cell senescence: hypertrophic arrest beyond restriction point,” J. Cell Physiol. 209: 592-97 (2006b).
12. Blagosklonny, “An anti-aging drug today: from senescence-promoting genes to anti-aging pill,” Drug Disc. Today 12: 218-24 (2007).
13. Caine et al., “Prolonged survival of pig orthotopic heart grafts treated with cyclosporin A,” Lancet 1: 1183-85 (1978).
14. Campisi, “Senescent cells, tumor suppression, and organismal aging: good citizens, bad neighbors,” Cell 120: 513-22 (2005).
15. Campistol et al., “Therapy after Early Cyclosporine Withdrawal Reduces the Risk for Cancer in Adult Renal Transplantation,” J. Am. Soc. Nephrol. 17: 581-89 (2006).
16. Carlson et al., “Rapamycin, a potential disease-modifying antiarthritic drug,” J. Pharmacol. Exp. Ther. 266: 1125-38 (1993).
17. Choi et al., “Expression of senescence-associated beta-galactosidase in enlarged prostates from men with benign prostatic hyperplasia,” Urology 56: 160-66 (2000).
18. Cullis et al., “Sirolimus-induced remission of posttransplantation lymphoproliferative disorder,” Am. J. Kidney Dis. 47: e67-72 (2006).
19. Elloso et al., “Protective effect of the immunosuppressant sirolimus against aortic atherosclerosis in apo E-deficient mice,” Am. J. Transplant. 3: 562-69 (2003).
20. Farrell et al., “Emerging therapies in multiple sclerosis,” Expert Opin. Emerg. Drugs 10: 797-816 (2005).
21. Foroncewicz et al., “Efficacy of rapamycin in patient with juvenile rheumatoid arthritis,” Transpl. Int. 18: 366-68 (2005).
22. Ha et al., “Attenuation of cardiac hypertrophy by inhibiting both mTOR and NFkappaB activation in vivo,” Free Radic. Biol. Med. 39: 1570-80 (2005).
23. Haider et al., “Resveratrol suppresses angiotensin II-induced Akt/protein kinase B and p70 S6 kinase phosphorylation and subsequent hypertrophy in rat aortic smooth muscle cells,” Mol. Pharmacol. 62: 772-77 (2002).
24. Harrington et al., “Restraining PI3K: mTOR signalling goes back to the membrane,” Trends Biochem. Sci. 30: 35-42 (2005).
25. Hayflick, “The future of ageing,” Nature 408: 267-69 (2000).
26. Jia et al., “The TOR pathway interacts with the insulin signaling pathway to regulate C. elegans larval development, metabolism and life span,” Development 131: 3897-906 (2004).
27. Kaeberlein et al., “Regulation of yeast replicative life span by TOR and Sch9 in response to nutrients,” Science 310: 1193-96 (2005).
28. Kahan, “Sirolimus: a ten-year perspective,” Transplant Proc. 36: 71-75 (2004).
29. Kapahi et al., “Regulation of lifespan in Drosophila by modulation of genes in the TOR signaling pathway,” Curr. Biol. 14: 885-90 (2004).
30. Kauffman et al., “Maintenance immunosuppression with target-of-rapamycin inhibitors is associated with a reduced incidence of de novo malignancies,” Transplantation 80: 883-89 (2005).
31. Khamzina et al., “Increased activation of the mammalian target of rapamycin pathway in liver and skeletal muscle of obese rats: possible involvement in obesity-linked insulin resistance,” Endocrinology 146: 1473-81 (2005).
32. Khurana et al., “TOR-mediated cell-cycle activation causes neurodegeneration in a Drosophila tauopathy model,” Curr. Biol. 16: 230-41 (2006).
33. Kimura et al., “daf-2, an insulin receptor-like gene that regulates longevity and diapause in Caenorhabditis elegans,” Science 277: 942-46 (1997).
34. Kneissel et al., “Everolimus suppresses cancellous bone loss, bone resorption, and cathepsin K expression by osteoclasts,” Bone 35: 1144-56 (2004).
35. Krtolica et al., “Senescent fibroblasts promote epithelial cell growth and tumorigenesis: a link between cancer and aging,” Proc. Natl. Acad. Sci. U.S.A. 98: 12072-77 (2001).
36. Le Bacquer et al., “Elevated sensitivity to diet-induced obesity and insulin resistance in mice lacking 4E-BP1 and 4E-BP2,” J. Clin. Invest. 117: 387-96 (2007).
37. Li et al., “Levels of mTOR and its downstream targets 4E-BP1, eEF2, and eEF2 kinase in relationships with tau in Alzheimer's disease brain,” FEBS J. 272: 4211-20 (2005).
38. Luong et al., “Activated FOXO-mediated insulin resistance is blocked by reduction of TOR activity,” Cell. Metab. 4: 133-42 (2006).
39. Manning, “Balancing Akt with S6K: implications for both metabolic diseases and tumorigenesis,” J. Cell. Biol. 167: 399-403 (2004).
40. Marsland et al., “The macrolide immunosuppressants in dermatology: mechanisms of action,” Eur. J. Dermatol. 12: 618-22 (2002).
41. Martel et al., “Inhibition of the immune response by rapamycin, a new antifungal antibiotic,” Can. J. Physiol. Pharmacol. 55: 48-51 (1977).
42. Mathew et al., “Two-year incidence of malignancy in sirolimus-treated renal transplant recipients: results from five multicenter studies,” Clin. Transplant. 18: 446-49 (2004).
43. Mohsin et al., “Complete regression of visceral Kaposi's sarcoma after conversion to sirolimus,” Exp. Clin. Transplant. 3: 366-69 (2005).
44. Morrisett et al., “Effects of sirolimus on plasma lipids, lipoprotein levels, and fatty acid metabolism in renal transplant patients,” J. Lipid Res. 43: 1170-80 (2002).
45. Nakamura et al., “Enhanced expression of p16 in seborrhoeic keratosis; a lesion of accumulated senescent epidermal cells in GI arrest,” Br. J. Dermatol. 149: 560-65 (2003).
46. Nungaray et al., “Rapamycin at six years can exhibit normal renal function without proteinuria or neoplasia after renal transplantation. A single-center experience,” Transplant Proc. 37: 3727-28 (2005).
47. Olshansky et al., “A potential decline in life expectancy in the United States in the 21st century,” N. Engl. J. Med. 352: 1138-45 (2005).
48. Ong et al., “mTOR as a potential therapeutic target for treatment of keloids and excessive scars,” Exp. Dermatol. 16: 394-404 (2007).
49. Oudit et al., “The role of phosphoinositide-3 kinase and PTEN in cardiovascular physiology and disease,” J. Mol. Cell. Cardiol. 37: 449-71 (2004).
50. Paccalin et al., “Activated mTOR and PKR Kinases in Lymphocytes Correlate with Memory and Cognitive Decline in Alzheimer's Disease,” Dement. Geriatr. Cogn. Disord. 22: 320-26 (2006).
51. Paolisso et al., “Low insulin resistance and preserved beta-cell function contribute to human longevity but are not associated with TH-INS genes,” Exp. Gerontol. 37: 149-56 (2001).
52. Powers et al., “Extension of chronological life span in yeast by decreased TOR pathway signaling,” Genes Dev. 20: 174-84 (2006).
53. Proud, “Ras, PI3-kinase and mTOR signaling in cardiac hypertrophy,” Cardiovasc. Res. 63: 403-13 (2004).
54. Rizell et al., “Impressive regression of primary liver cancer after treatment with sirolimus,” Acta. Oncol. 44: 496 (2005).
55. Rodriguez et al., “Oral Rapamycin After Coronary Bare-Metal Stent Implantation to Prevent Restenosis The Prospective, Randomized Oral Rapamycin in Argentina (ORAR II) Study,” J. Am. Coll. Cardiol. 47: 1522-29 (2006).
56. Rubinsztein, “The roles of intracellular protein-degradation pathways in neurodegeneration,” Nature 443: 780-86 (2006).
57. Ruggero et al., “The translation factor eIF-4E promotes tumor formation and cooperates with c-Myc in lymphomagenesis,” Nat. Med. 10: 484-86 (2004).
58. Sakaguchi et al., “Inhibition of mTOR signaling with rapamycin attenuates renal hypertrophy in the early diabetic mice,” Biochem. Biophys. Res. Commun. 340: 296-301 (2006).
59. Sarraj et al., “Reconstitution of DNA synthetic capacity in senescent normal human fibroblasts by expressing cellular factors E2F and Mdm2,” Exp. Cell. Res. 270: 268-76 (2001).
60. Shah et al., “Inappropriate activation of the TSC/Rheb/mTOR/S6K cassette induces IRS1/2 depletion, insulin resistance, and cell survival deficiencies,” Curr. Biol. 14: 1650-56 (2004).
61. Sharp et al., “Evidence for down-regulation of phosphoinositide 3-kinase/Akt/mammalian target of rapamycin (PI3K/Akt/mTOR)-dependent translation regulatory signaling pathways in Ames dwarf mice,” J. Gerontol. A. Biol. Sci. Med. Sci. 60: 293-300 (2005).
62. Shaw et al., “The kinase LKB1 mediates glucose homeostasis in liver and therapeutic effects of metformin,” Science 310: 1642-46 (2005).
63. Stallone et al., “Sirolimus for Kaposi's sarcoma in renal-transplant recipients,” N. Engl. J. Med. 352: 1317-23 (2005).
64. Thomson et al., “Immunosuppressive properties of FK-506 and rapamycin,” Lancet 2: 443-44 (1989).
65. Tzatsos et al., “Nutrients suppress phosphatidylinositol 3-kinase/Akt signaling via raptor-dependent mTOR-mediated insulin receptor substrate 1 phosphorylation,” Mol. Cell. Biol. 26: 63-76 (2006).
66. Um et al., “Absence of S6K1 protects against age- and diet-induced obesity while enhancing insulin sensitivity,” Nature 431: 200-05 (2004).
67. van Heemst et al., “Reduced insulin/IGF-1 signalling and human longevity,” Aging Cell 4: 79-85 (2005).
68. Vellai et al., “Genetics: influence of TOR kinase on lifespan in C. elegans,” Nature 426: 620 (2003).
69. Vezina et al., “Rapamycin (AY-22,989), a new antifungal antibiotic. I. Taxonomy of the producing streptomycete and isolation of the active principle,” J. Antibiot. (Tokyo) 28: 721-26 (1975).
70. Yakupoglu et al., “Individualization of Immunosuppressive Therapy. III. Sirolimus Associated With a Reduced Incidence of Malignancy,” Transplant Proc. 38: 358-61 (2006).
71. Zhang et al., “PDGFRs are critical for PI3K/Akt activation and negatively regulated by mTOR,” J. Clin. Invest. 117: 730-38 (2007).
72. Zhang et al., “Loss of Tsc1/Tsc2 activates mTOR and disrupts PI3K-Akt signaling through downregulation of PDGFR,” J. Clin. Invest. 112: 1223-33 (2003).
73. Zmonarski et al., “Regression of Kaposi's sarcoma in renal graft recipients after conversion to sirolimus treatment,” Transplant Proc. 37: 964-66 (2005).

1-22. (canceled)

23. A method of inhibiting senescence in mammalian cells comprising exposing the mammalian cells to an inhibitor of TOR.

24. The method of claim 23, wherein senescence is inhibited in mammalian cells prior to transplantation of the mammalian cells into a mammal.

25. A method of inhibiting senescence in an organism comprising administering a therapeutically effective amount of an inhibitor of TOR to the organism.

26. A method for treating or preventing an age-related disease, condition, or disorder comprising administering a therapeutically effective amount of an inhibitor of TOR to a patient in need thereof.

27. The method of claim 26, wherein the age-related disease, condition, or disorder is insulin resistance, benign prostatic hyperplasia, hearing loss, osteoporosis, age-related macular degeneration, Alzheimer's disease, or Parkinson's disease.

28. The method of claim 27, wherein the age-related disease, condition, or disorder is benign prostatic hyperplasia.

29. The method of any of claim 23, 25, or 26, wherein the inhibitor of TOR is rapamycin or an analog of rapamycin.

30. The method of claim 29, wherein the analog of rapamycin is everolimus, tacrolimus, CCI-779, ABT-578, AP-23675, AP-23573, AP-23841, 7-epi-rapamycin, 7-thiomethyl-rapamycin, 7-epi-trimethoxyphenyl-rapamycin, 7-epi-thiomethyl-rapamycin, 7-demethoxy-rapamycin, 32-demethoxy-rapamycin, 2-desmethyl-rapamycin, or 42-O-(2-hydroxy)ethyl rapamycin.

31. The method of any of claim 23, 25, or 26, wherein the inhibitor of TOR is administered with a second compound.

32. The method of claim 31, wherein the second compound is selected from the group consisting of vitamin E, vitamin A, an antibacterial antibiotic, an antioxidant, L-carnitine, lipoic acid, metformine, resveratrol, leptine, a non-steroid anti-inflammatory drug, and a COX inhibitor.
